(19) United States
(12) Patent Application Publication
(10) Pub. No.: US 2005/0010132 A1
(43) Pub. Date: Jan. 13, 2005

(54) Swab having easily displaceable tip for collecting a saliva sample and inserting it into a collection device, and method of using same

(76) Inventors: Dale Pestes, Gresham, OR (US); Larry Pestes, Boring, OR (US)

Correspondence Address: Chernoff Vilhauer McClung & Stenzel, L.L.P., 1600 ODS Tower, 601 SW Second Avenue, Portland, OR, 97204-3157, US

(21) Appl. No.: 10/615,712

(22) Filed: Jul. 8, 2003

(51) Int. Cl. A61B 10/00

(52) U.S. Cl. 600/572

(57) ABSTRACT

A swab for collecting specimens from patients has an elongate handle with an absorbent fabric swab tip at one end. The swab tip is releasably attached to the handle, and a sleeve, which slidably fits on the handle, can be pushed against the swab tip after a specimen has been collected to displace the swab tip from the handle and deposit it into a collection device.

### BACKGROUND AND SUMMARY OF THE INVENTION

Swabs, having an elongate rigid handle and a soft, absorbent fabric or cotton tip are well-known in the medical art. In the past these swabs have been used to collect a sample from a patient for testing. However, in many cases the sample must be deposited into a collection device, such as a test tube, which generally is too small to receive the entire swab. Accordingly, it is common to collect some types of samples, such as saliva samples, using a specimen collection probe having an elongate hollow handle with a slot opening out of the hollow center at one end. A flat collection head fits tightly in this slot and projects outwardly from the handle. A rod slidably fits in the handle and when pushed toward the end of the handle which carries the collection head the rod displaces the collection head from the handle and deposits it into a collection device. These prior art specimen collection probes are sold by Saliva Diagnostic Systems of Medford, N.Y., under the OMNI-SWAB trademark. Because of the separate displaceable collection head, the prior art probes are relatively expensive to make and the collection head can inadvertently be displaced while a specimen is being obtained.

The subject invention overcomes the foregoing shortcomings of the prior art specimen collection probes by providing a swab having an elongate handle with an absorbent fabric swab tip attached to one end. A sleeve slidably fits on the handle and is moveable along the handle so that it can be pushed against the swab tip to displace the swab tip from the handle and into a collection container.

The foregoing and other objectives, features, and advantages of the invention will be more readily understood upon consideration of the following detailed description of the invention, taken in conjunction with the accompanying drawings.

### BRIEF DESCRIPTION OF THE SEVERAL VIEWS OF THE DRAWINGS

FIG. 1 is a side elevation view showing a swab embodying the subject invention being used to obtain a specimen from a patient.

FIG. 2 is a side elevation view showing the swab in position to deposit the swab head into a collection container after the specimen has been collected.

FIG. 3 is a side elevation view showing the swab tip displaced from the swab handle and deposited into the collection container.

### DETAILED DESCRIPTION OF THE PREFERRED EMBODIMENT

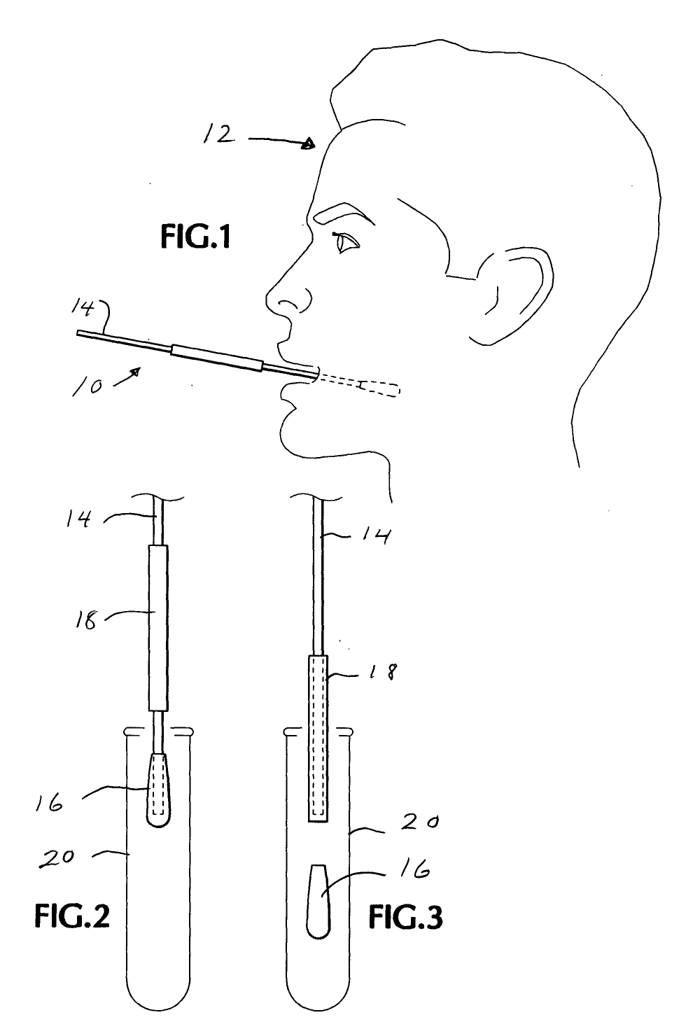

Referring to FIG. 1 of the drawings, a swab 10 is used to collect a specimen from a patient 12. In the drawings the swab is shown as being used to collect a saliva specimen. The swab includes an elongate handle 14. In the embodiment illustrated the handle is circular in cross-section but it could have other shapes as well. The handle needs to be relatively rigid and preferably is made from polystyrene. In a preferred embodiment the handle is approximately 5½ inches long, is hollow, has an outside diameter of approximately 0.100 inches and an inside diameter of 0.055 inches. The dimensions of the handle and the material it is made from are not critical and any common swab handle would suffice.

Located on a first end of the handle is an absorbent fabric swab tip 16. In a preferred embodiment the swab tip 16 is made by wrapping a thin strip of Dacron onto the end of the handle. The Dacron material is kept under tension as it is wrapped onto the handle. After the Dacron material is would onto the handle it is clamped into a heated mold to shape it and bind it together in a tear-drop shape. With one exception the swab tip is fabricated the same way as normal swab tips are, and other known methods of making swab tips will work as well. The difference between the swab tip of the subject invention and the prior art swab tips is that no glue or other binder is used and the swab tip is maintained on the handle entirely by friction created from winding the Dacron material onto the handle while it is under tension and by whatever adhesive effect is achieved due to the application of heat and pressure. Thus, the swab tip can be easily removed from the handle but will remain firmly in place on the handle during normal use.

Located on the handle 14 below the swab tip 16 is a sleeve 18. In the embodiment illustrated the sleeve is annular in cross-section with a 0.158 inch outside diameter and an inside diameter which allows it to be slid along the handle when pressure is applied but does not allow it to move along the handle merely due to movement of the swab 10. The sleeve 18 needs to be long enough to be easily gripped by the user of the swab but not so long as to prevent the user from easily grasping the portion of the handle not covered by the swab tip. In the embodiment illustrated the handle covers slightly less than half of the portion of the handle that is not covered by the swab tip.

In use, the swab 10 is manipulated to collect a specimen on the swab tip 16 in the standard manner, FIG. 1. The swab is then moved to where the swab tip 16 is located above or in an open top device, such as test tube 20, FIG. 2. The sleeve 18 is then urged toward the first end of the handle 14 to displace the swab tip from the handle and into the collection device. The handle is then disposed of and the swab tip and test tube are sent for processing.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A swab for collecting a specimen and depositing the specimen into a collection device comprising:
(a) an elongate handle;
(b) a swab tip which is releasably attached to said handle, at a first end thereof; and
(c) a sleeve which slidably fits on said handle and is moveable such that it can be urged toward said first end of said handle to displace said swab tip off of said handle.

2. The swab of claim 1 wherein said handle is circular in cross-section and said sleeve is annular in cross-section.

3. The swab of claim 1 wherein said swab tip is an absorbent fabric material.

4. The swab of claim 3 wherein said swab tip is made from a strip of Dacron material.

5. The swab tip of claim 4 wherein said swab tip is secured to send handle only by friction created by winding said strip of Dacron material onto said handle while said strip is in tension.

6. A method of collecting a specimen with a swab and depositing a specimen into a collection device comprising:
(a) providing a swab having a swab tip which is releasably attached to one end of an elongate handle;
(b) providing a sleeve which is slidable along said handle;
(c) collecting a specimen on said swab tip;
(d) placing said swab such that said swab tip is adjacent to an opening in a collection device; and
(e) urging said sleeve along said handle and against said swab tip to displace said swab tip from said handle and into said collection device.
